(19) United States
(12) Patent Application Publication
(10) Pub. No.: US 2005/0233003 A1
(43) Pub. Date: Oct. 20, 2005

(54) Hydrophilic dispersions of nanoparticles of inclusion complexes of salicylic acid

(75) Inventors: Rina Goldshtein, Har Hebron (IL); Vadim Goldshtein, Har Hebron (IL); Vladimir Mikunis, Raanana (IL); Larisa Gitis, Holon (IL)

Correspondence Address: BROWDY AND NEIMARK, P.L.L.C.;624 NINTH STREET, NW, SUITE 300, WASHINGTON, DC, 20001-5303, US

(73) Assignee: SoluBest Ltd., Rehovot (IL)

(21) Appl. No.: 11/100,623

(22) Filed: Apr. 7, 2005

Related U.S. Application Data
(63) Continuation-in-part of application No. 10/952,380, filed on Sep. 29, 2004.
(63) Continuation-in-part of application No. 10/256,023, filed on Sep. 26, 2002.
Application No. 10/256,023 is a continuation-in-part of application No. 09/966,847, filed on Sep. 28, 2001, now Pat. No. 6,878,693.
(60) Provisional application No. 60/507,623, filed on Sep. 30, 2003.

(51) Int. Cl. A61K 31/60; A61K 9/48; A61K 9/16; A61K 9/50

(52) U.S. Cl. 424/490; 514/159

(57) ABSTRACT

The invention provides a hydrophilic inclusion complex consisting essentially of nanosized particles of salicylic acid wrapped in an amphiphilic polymer, wherein said amphiphilic polymer is selected from the group consisting of polyacrylic acid, polyacrylamide and copolymers thereof, polymethacrylamide and copolymers thereof, and polylysine, and said amphiphilic polymer is modified by reaction with urea or a derivative thereof, nicotinamide or guanidine. Further provided are hydrophilic dispersions comprising nanoparticles of said inclusion complexes and pharmaceutical and cosmetic compositions comprising said dispersions.

### CROSS-REFERENCE TO RELATED APPLICATIONS

The present application is a continuation-in-part of application Ser. No. No. 10/952,380, filed Sep. 29, 2004, which is a non-provisional of the Provisional Application No. 60/507,623, filed Sep. 30, 2003 and a continuation-in-part of application Ser. No. 10/256,023, filed Sep. 26, 2002, which is a continuation-in-part of application Ser. No. 09/966,847, filed Sep. 28, 2001, the entire contents of each and all these applications being hereby incorporated by reference herein in their entirety as if fully disclosed herein.

### FIELD OF THE INVENTION

The present invention is in the field of nanoparticles. More particularly, the invention relates to soluble nanosized particles consisting of inclusion complexes of salicylic acid surrounded by and entrapped within suitable amphiphilic polymers, and methods of producing such salicylic acid nanoparticles.

### BACKGROUND OF THE INVENTION

Two formidable barriers to effective drug delivery and hence to disease treatment, are solubility and stability. To be absorbed in the human body, a compound has to be soluble in both water and fats (lipids). Solubility in water is, however, often associated with poor fat solubility and vice-versa.

Over one third of drugs listed in the U.S. Pharmacopoeia and about 50% of new chemical entities (NCEs) are insoluble or poorly insoluble in water. Over 40% of drug molecules and drug compounds are insoluble in the human body. In spite of this, lipophilic drug substances having low water solubility are a growing drug class having increasing applicability in a variety of therapeutic areas and for a variety of pathologies.

Solubility and stability issues are major formulation obstacles hindering the development of therapeutic agents. Aqueous solubility is a necessary but frequently elusive property for formulations of the complex organic structures found in pharmaceuticals. Traditional formulation systems for very insoluble drugs have involved a combination of organic solvents, surfactants and extreme pH conditions. These formulations are often irritating to the patient and may cause adverse reactions.

The size of the drug molecules also plays a major role in their solubility and stability as well as bioavailability. Bioavailability refers to the degree to which a drug becomes available to the target tissue or any alternative in vivo target (i.e., receptors, tumors, etc.) after being administered to the body. Poor bioavailability is a significant problem encountered in the development of pharmaceutical compositions, particularly those containing an active ingredient that is poorly soluble in water. Poorly water-soluble drugs tend to be eliminated from the gastrointestinal tract before being absorbed into the circulation. It is known that the rate of dissolution of a particulate drug can increase with increasing surface area, that is, decreasing particle size

Recently, there has been an explosion of interest in nanotechnology, the manipulation on the nanoscale. Nanotechnology is not an entirely new field: colloidal sols and supported platinum catalysts are nanoparticles. Nevertheless, the recent interest in the nanoscale has produced, among numerous other things, materials used for and in drug delivery. Nanoparticles are generally considered to be solids whose diameter varies between 1-1000 nm.

Although a number of solubilization technologies do exist, such as liposomes, cylcodextrins, microencapuslation, and dendrimers, each of these technologies has a number of significant disadvantages.

Liposomes, as drug carriers, have several potential advantages, including the ability to carry a significant amount of drug, relative ease of preparation, and low toxicity if natural lipids are used. However, common problems encountered with liposomes include: low stability, short shelf-life, poor tissue specificity, and toxicity with non-native lipids. Additionally, the uptake by phagocytic cells reduces circulation times. Furthermore, preparing liposome formulations that exhibit narrow size distribution has been a formidable challenge under demanding conditions, as well as a costly one. Also, membrane clogging often results during the production of larger volumes required for pharmaceutical production of a particular drug.

Cyclodextrins are crystalline, water-soluble, cyclic, non-reducing oligo-saccharides built from six, seven, or eight glucopyranose units, referred to as alpha, beta and gamma cyclodextrin, respectively, which have long been known as products that are capable of forming inclusion complexes. The cyclodextrin structure provides a molecule shaped like a segment of a hollow cone with an exterior hydrophilic surface and interior hydrophobic cavity. The hydrophilic surface generates good water solubility for the cyclodextrin and the hydrophobic cavity provides a favorable environment in which to enclose, envelope or entrap the drug molecule. This association isolates the drug from the aqueous solvent and may increase the drug's water solubility and stability.

For a long time, most cyclodextrins had been no more than scientific curiosities due to their limited availability and high price, but lately cyclodextrins and their chemically modified derivatives became available commercially, generating a new technology of packing on the molecular level. Cyclodextrins are, however, fraught with disadvantages including limited space available for the active molecule to be entrapped inside the core, lack of pure stability of the complex, limited availability in the marketplace, and high price.

Microencapsulation is a process by which tiny parcels of a gas, liquid, or solid active ingredient (“core material”) are packaged within a second material for the purpose of shielding the active ingredient from the surrounding environment. These capsules, which range in size from one micron (one-thousandth of a millimeter) to approximately seven millimeters, release their contents at a later time by means appropriate to the application.

There are four typical mechanisms by which the core material is released from a microcapsule: (1) mechanical rupture of the capsule wall, (2) dissolution of the wall, (3) melting of the wall, and (4) diffusion through the wall. Less common release mechanisms include ablation (slow erosion of the shell) and biodegradation.

Microencapsulation covers several technologies, where a certain material is coated to obtain a micro-package of the active compound. The coating is performed to stabilize the material, for taste masking, preparing free flowing material of otherwise clogging agents etc. and many other purposes. This technology has been successfully applied in the feed additive industry and to agriculture. The relatively high production cost needed for many of the formulations is, however, a significant disadvantage.

In the cases of nanoencapsulation and nanoparticles (which are advantageously shaped as spheres and, hence, nanospheres), two types of systems having different inner structures are possible: (i) a matrix-type system composed of an entanglement of oligomer or polymer units, defined as nanoparticles or nanospheres, and (ii) a reservoir-type system, consisting of an oily core surrounded by a polymer wall, defined as a nanocapsule.

Depending upon the nature of the materials used to prepare the nanospheres, the following classification exists: (a) amphiphilic macromolecules that undergo a cross-linking reaction during preparation of the nanospheres; (b) monomers that polymerize during preparation of the nanoparticles; and (c) hydrophobic polymers, which are initially dissolved in organic solvents and then precipitated under controlled conditions to produce nanoparticles.

Problems associated with the use of polymers in micro- and nanoencapsulation include the use of toxic emulgators in emulsions or dispersions, polymerization or the application of high shear forces during emulsification process, insufficient biocompatibility and biodegradability, balance of hydrophilic and hydrophobic moieties, etc. These characteristics lead to insufficient drug release.

Dendrimers are a class of polymers distinguished by their highly branched, tree-like structures. They are synthesized in an iterative fashion from ABn monomers, with each iteration adding a layer or “generation” to the growing polymer. Dendrimers of up to ten generations have been synthesized with molecular weights in excess of 106 kDa. One important feature of dendrimeric polymers is their narrow molecular weight distributions. Indeed, depending on the synthetic strategy used, dendrimers with molecular weights in excess of 20 kDa can be made as single compounds.

Dendrimers, like liposomes, display the property of encapsulation, and are able to sequester molecules within the interior spaces. Because they are single molecules, not assemblies, drug-dendrimer complexes are expected to be significantly more stable than liposomal drugs. Dendrimers are thus considered as one of the most promising vehicles for drug delivery systems. However, the dendrimer technology is still in the research stage, and it is speculated that it will take years before it is applied in the industry as an efficient drug delivery system.

Salicylic acid or 2-hydroxybenzoic acid, is a colorless, crystalline organic carboxylic acid soluble in ethanol, ether, and in lipids (oil), but only slightly soluble in water. The medical benefits of salicylic acid, including its anti-inflammatory and anti-microbial effects, and its benefits in cosmetic and dermatological formulations as an exfoliant and for treatment of dandruff, acne, skin wrinkling, skin pigmentation, warts, freckles and other skin-related conditions and for UV protection, are well known. Salicylic acid solubility and physical state are central factors for determining the compound's efficacy.

A number of publications disclose means for increasing salicylic acid solubility. For example, U.S. Pat. No. 5,328,690 discloses polyamino acid dispersants used in cosmetic products such as shampoos for dispersing agents such as salicylic acid as anti-dandruff agent.

U.S. Pat. No. 5,942,501 discloses a complex of salicylic acid and derivatized β-cyclodextrin which increases the solubility of salicylic acid in water or in mixed aqueous solvents from 0.3 up to 8%.

U.S. Pat. No. 6,669,964 discloses a composition for solubilizing and stabilizing salicylic acid for use in an anhydrous liquid precursor solution for use in the formulation of dermatological, cosmetic, toiletry and personal care products. The anhydrous liquid composition comprises butylene glycol and glycereth-26 acting as solubilizer agents.

U.S. Pat. No. 6,623,761 discloses a method of making nanoparticles of substantially water-insoluble therapeutic agents. Salicylic acid is mentioned as one of the suitable therapeutic agents, but nanoparticles of salicylic acid are not disclosed therein.

U.S. patent application Publication 2003/0143166 discloses an aqueous dispersion of sparingly water-soluble or water-insoluble organic UV filter substances, including salicylic acid, in which the substance is in a colloidally disperse phase in amorphous or partially amorphous form. The particles of the colloidally disperse phase may comprise a water-insoluble polymer matrix into which the sparingly water-soluble or water-insoluble organic substance has been embedded. Similarly, U.S. patent application Publication 2004/0247542 discloses a dispersion and a cosmetic comprising an ultraviolet light scattering agent, e.g. salicylic acid, coated with an inorganic oxide, and a dispersant which is a water soluble polymer, e.g. polyacrylamide.

There is a need to provide salicylic acid preparations that are biocompatible and stable and efficiently deliver salicylic acid that can be used as a food additive and in the cosmetic, dermatological and other pharmaceutical fields.

### SUMMARY OF THE INVENTION

It is an object of the present invention to provide a stable and solubilized salicylic acid dispersion useful for incorporation into cosmetic and dermatologic preparations for an increased efficacy of the product in treating and preventing skin-related problems, such as dandruff, acne, skin wrinkling, skin pigmentation, warts, freckles and the like.

It is another object of the present invention to provide a stabilized dispersion of nanoparticles comprising salicylic acid, which does not form crystals on standing and remains stable with respect to disordered crystallinity.

It is a further object of the present invention to provide a stabilized and solubilized salicylic acid solution having an increased bioavailability of salicylic acid compound for an increased efficacy in treating and preventing skin-related problems when used in a cosmetic and dermatologic formulation.

The present invention relates to a hydrophilic inclusion complex consisting essentially of nanosized particles of salicylic acid wrapped in an amphiphilic polymer such that non-valent bonds are formed between the salicylic acid and the amphiphilic polymer, wherein said amphiphilic polymer is selected from the group consisting of polyacrylic acid, polyacrylamide and copolymers thereof, polymethacrylamide and copolymers thereof, and polylysine, and said amphiphilic polymer is modified by reaction with urea or a derivative thereof, nicotinamide or guanidine.

The invention further relates to hydrophilic dispersions comprising nanoparticles of the said inclusion complexes of salicylic acid and to stable pharmaceutical and cosmetic compositions comprising said dispersions.

### BRIEF DESCRIPTION OF THE DRAWINGS

FIG. 1 depicts light scattering measurements of the sizes of nanoparticles of salicylic acid, that were prepared by the one-step solvent-free method using 0.2% polyacrylamide (PAA) modified with 2.1% urea, as described in Example 4. The analysis was performed using a Zetasizer Nano light scattering technique (a resolution of 0.6-6000 nm) with a 1:10 dilution of the samples.

FIG. 2 is a photograph of a cryo-transmission electron microscopy (TEM) analysis, showing nanoparticles of salicylic acid that were prepared by the two-step solvent-free method using 0.2% PAA modified with 2.2% urea, as described in Example 3.

FIGS. 3A-3D show the Fourier transform infrared (FTIR) spectroscopy analysis of pure salicylic acid (FIG. 3A), sodium salicylate salt (FIG. 3B) and nanoparticles of inclusion complexes of salicylic acid (6.23% and 6.48%) which were prepared by the two-step and one-step organic solvent-free processes, respectively (FIGS. 3C and 3D, respectively).

FIG. 4 is a graph showing the release of salicylic acid from inclusion complexes prepared using the two-step organic solvent-free process described in Example 3 herein, following a dialysis. Dialysis was performed using dialysis tubing having a pore size of either 3500 Daltons (MW 3500) or 7000 Daltons (MW 7000), filled with 2 ml of a dispersion comprising nanoparticles of salicylic acid having a final salicylic acid concentration of 7%. At the indicated times, 1 ml aliquots were removed from the external solution for analysis of the salicylic acid content by reverse-phase high-pressure liquid chromatography (RP-HPLC). Measurement of the salicylic acid in each experimental sample was followed by calculation of the salicylic acid concentration according to the area of salicylic acid peak in that sample.

### DETAILED DESCRIPTION OF THE INVENTION

The present invention provides nanoparticles and methods for the production of soluble nanoparticles and, in particular, hydrophilic dispersions of nanoparticles of inclusion complexes of salicylic acid in certain amphiphilic polymers.

The soluble nanoparticles, referred to herein sometimes as “solu-nanoparticles” or “solumers”, are differentiated by the use of water-soluble amphiphilic polymers that are capable of producing molecular complexes with lipophilic and hydrophilic active compounds or molecules (particularly, drugs and pharmaceuticals). The solu-nanoparticles formed in accordance with the present invention render the water-insoluble salicylic acid soluble in water. In addition, in this form the salicylic acid is readily bioavailable in the human body.

As used herein, the term “inclusion complex” refers to a complex in which one component—the amphiphilic polymer (the “host), forms a cavity in which molecular entities of a second chemical species—the salicylic acid in the present invention (the “guest”), are located. Thus, in accordance with the present invention, inclusion complexes are provided in which the host is the amphiphilic polymer and the guest is the salicylic acid molecules wrapped and fixated or secured within the cavity or space formed by said amphiphilic polymer host.

In accordance with the present invention, the inclusion complexes contain the salicylic acid molecules, which interact with the polymer by non-valent interactions and form a polymer-salicylic acid as a distinct molecular entity. A significant advantage and unique feature of the inclusion complex of the present invention is that no new chemical bonds are formed and no existing bonds are destroyed during the formation of the inclusion complex. The particles comprising the inclusion complexes are nanosized, and no change occurs in the salicylic acid molecule itself when it is enveloped, or advantageously wrapped, by the polymer.

Other important characteristic of the inclusion complex of the invention is that the salicylic acid may be presented in a non-crystalline state. As used herein, the term “non-crystalline” state is intended to include both disordered crystalline and, preferably, amorphous state.

The creation of the complex does not involve the formation of any valent bonds (which may change the characteristics or properties of the active compound). As used herein, the term “non-valent” is intended to refer to non-covalent, non-ionic and non-semi-polar bonds and/or interactions and includes weak, non-covalent bonds and/or interactions such as electrostatic forces, hydrogen bonds and Van der Waals forces formed during the creation of the inclusion complex. The formation of non-valent bonds preserves the structure and properties of the salicylic acid.

The solubilized salicylic acid nanoparticles of the invention remain stable for long periods of time, may be manufactured at a low cost, and may improve the overall bioavailability of the salicylic acid.

In one aspect, the present invention relates to a hydrophilic inclusion complex consisting essentially of nanosized particles of salicylic acid wrapped in an amphiphilic polymer such that non-valent bonds are formed between the salicylic acid and the amphiphilic polymer, wherein said amphiphilic polymer is selected from the group consisting of polyacrylic acid, polyacrylamide and copolymers thereof, polymethacrylamide and copolymers thereof, and polylysine, and said amphiphilic polymer is modified by reaction with urea or a derivative thereof, nicotinamide or guanidine.

In one embodiment, the amphiphilic polymer is polyacrylic acid. In another embodiment, the polymer is polyacrylamide. In a further embodiment, the polymer is polymethacrylamide. In a yet another embodiment, the polymer is polylysine.

In a still another embodiment, the amphiphilic polymer is a copolymer of acrylamide or methacrylamide with one or two monomers selected from the group consisting of acrylic acid, methacrylic acid, an alkyl acrylate, an alkyl methacrylate, acrylonitrile, ethyleneimine, vinyl acetate, styrene, maleic anhydride and vinyl pyrrolidone. The alkyl radical of the alkyl acrylate or alkyl methacrylate monomer may be a straight, branched or cyclic unsubstituted (C₁-C₁₂)alkyl, such as, but not limited to, methyl acrylate, ethyl acrylate, butyl acrylate, n-octyl acrylate, 2-ethylhexyl acrylate or cyclohexyl methacrylate, or the alkyl may be substituted by a radical selected from the group consisting of OH, —CONH₂, —NH₂, —COOH, —SO₃H, and —PO₃H₂. In a preferred embodiment, the alkyl is substituted by hydroxyl and includes, without being limited to, 2-hydroxyethyl acrylate, 2-hydroxyethyl methacrylate, or 2-hydroxypropyl methacrylate.

In accordance with the present invention, the amphiphilic polymer is modified by reaction with urea or a derivative thereof, nicotinamide or guanidine.

In one embodiment, the modifier of the amphiphilic polymer is urea. In another embodiment, the modifier is a urea derivative selected from the group consisting of methylol urea, acetyl urea, semicarbazide and thiosemicarbazide. In a further embodiment, the modifier is nicotinamide or guanidine.

In a preferred embodiment of the invention, the amphiphilic polymer is polyacrylamide modified by reaction with urea. In another embodiment, the amphiphilic polymer is polymethacrylamide modified by reaction with urea.

In another aspect, the present invention relates to a hydrophilic dispersion comprising nanoparticles of inclusion complexes of salicylic acid wrapped in an amphiphilic polymer such that non-valent bonds are formed between the salicylic acid and the amphiphilic polymer, wherein said amphiphilic polymer is selected from the group consisting of polyacrylic acid, polyacrylamide and copolymers thereof, polymethacrylamide and copolymers thereof, and polylysine, and said amphiphilic polymer is modified by reaction with urea or a derivative thereof, nicotinamide or guanidine.

The size of the nanoparticles in the hydrophilic dispersion are in a range from approximately 1 to approximately 1000, preferably from approximately 10 to approximately 100 nanometers.

In another aspect, the present invention relates to a method using an organic solvent for the preparation of a dispersion of the invention, which comprises:
(i) preparation of a solution of the amphiphilic polymer in water;
(ii) modification of the amphiphilic polymer by reaction with urea or a derivative thereof, nicotinamide or guanidine, under heat and pressure;
(iii) preparation of a molecular solution of salicylic acid in an organic solvent;
(iv) dripping the cold organic solution (iii) of the salicylic acid into the modified polymer water solution (ii), while heating at a temperature 5-10° C. above the boiling point of the organic solvent of (iii), under constant mixing, thus causing evaporation of the organic solvent; and
(v) subjecting the dispersion obtained in (iv) to autoclave treatment, thus obtaining the desired dispersion comprising nanoparticles of inclusion complexes of salicylic acid entrapped within said amphiphilic polymer.

The organic solvent for the dissolution of the salicylic acid may be methyl acetate or dichloromethane.

According to this method, while evaporation of the organic solvent occurs in step (iv), particles of salicylic acid remain in the dispersed phase. The polymer molecule in the polymer solution then surrounds or envelopes, and more appropriately, wraps, the salicylic acid that had remained in the particles of the dispersed phase after evaporation of the solvent, thus forming a homogeneous nano-sized dispersion of solubilized salicylic acid wrapped by the amphiphilic polymer in an inclusion complex.

In another aspect, the present invention provides a solvent-free process for the preparation of the dispersions of the invention. In one-embodiment, the solvent-free process is a two-step process, comprising:
(i) preparation of a solution of the amphiphilic polymer in water;
(ii) modification of the amphiphilic polymer by reaction with urea or a derivative thereof, nicotinamide or guanidine, under heat and pressure, in an autoclave;
(iii) addition of salicylic acid powder to the modified polymer water solution; and
(iv) subjecting the dispersion obtained in (iii) to autoclave treatment, thus obtaining the desired dispersion comprising nanoparticles of inclusion complexes of salicylic acid entrapped within said modified amphiphilic polymer.

In another embodiment, the invention relates to a one-step solvent-free process for the preparation of the dispersions of the invention, comprising:
(i) preparation of a solution of the amphiphilic polymer in water;
(iii) modification of the amphiphilic polymer by reaction with urea or a derivative thereof, nicotinamide or guanidine;
(iii) addition of salicylic acid powder to the modified polymer water solution; and
(iv) subjecting the dispersion obtained in (iii) to autoclave treatment, thus obtaining the desired hydrophilic dispersion comprising nanoparticles of inclusion complexes of salicylic acid entrapped within said amphiphilic polymer.

The hydrophilic dispersion contains water-soluble nanoparticles of salicylic acid and may be also designated a nanodispersion and is, in fact, a fine dispersion of the nanoparticles that may have the appearance of a solution, but is not a classical aqueous solution. The nanodispersion is stable, meaning that it is a stable fine dispersion of the nanoparticles. The stability of the nanoparticles and of the inclusion complexes has more than one meaning. The nanoparticles should be stable as part of a nanocomplex over time, while remaining in the dispersion media. In fact, the nanodispersions are stable over time without separation of phases. Furthermore, any non-crystalline, preferably amorphous, state, should be also retained over time.

It is worth noting that in the process used in the present invention, the components of the system do not result in micelles nor do they form classical dispersion systems.

In addition, after dispersion of the salicylic acid to nanosize and fixation by the polymers to form an inclusion complex, enhanced solubility in physiological fluids, in vivo, improved absorption, and improved biological activity, as well as transmission to a stable non-crystalline state, are achieved.

In most preferred embodiments of the present invention, not less than 80% of the nanoparticles in the nanodispersion are within the size range, when the size deviation is not greater than 20%, and the particle size is within the nano range, namely less than 1000 nm, preferably 100 nm or even less.

In another aspect, the present invention provides a stable pharmaceutical composition comprising a pharmaceutically acceptable carrier and a hydrophilic dispersion of salicylic acid nanoparticles according to the invention. The pharmaceutical composition is useful for all pharmaceutical uses known or to be discovered for salicylic acid, in particular for dermatological uses.

In a further aspect, the present invention provides a stable cosmetic composition comprising a hydrophilic dispersion of salicylic acid nanoparticles of the invention, useful in cosmetic, toiletry and personal care products.

The invention will now be illustrated by the following non-limiting examples.

### EXAMPLES

### Example 1

### Preparation of Urea-Modified Polyacrylamide (PAA)

Various conditions were used for preparing the polymer solutions of polyacrylamide (PAA) and urea-modified-PAA, as shown in Table 1. The unmodified PAA was used in some experiments but did not provide good results.

Specifically, 3.3 (or 1) grams of PAA (CAS Number 9003058; Acros Organic, N.J., USA) were dissolved per liter water by thorough stirring, while heating at 60-90° C. (preferably 80-90° C.) for 80-120 min. Then, 2.1-4 grams of urea were dissolved per liter of the resulting solution by thorough stirring. The mixture was heated to above 100° C. (110-125° C.) under pressure (up to 2 atm) in an autoclave for about 80 minutes, in order to complete the reaction between the polymer and the urea. The resulting pH and viscosity of this and the other solutions prepared by variations on this process were measured and are presented in Table 1. The solution was cooled to room temperature before proceeding to the step in which the inclusion complexes are formed. Regardless of the amount of urea added within the range noted above and of PAA heating time, the quality of the resulting polymeric solution was generally robust as long as urea treatment was done. These conditions yielded a solution with an average pH of 9.2 (general range between 9.03-9.37) and an average viscosity of 38.4 cP (general range between 29.4-52.8 cP, except in one instance). As expected, 0.1% PAA solutions had lower viscosities than 0.33% PAA solutions. When no urea was added, or when urea was added, but the autoclaving step was omitted, the average pH of the resulting solution was 6.56 (5.98-7.37), and the average viscosity was 16.6 cP (6-29 cP). Therefore, the autoclaving step was considered necessary for preparing urea-modified PAA.

TABLE 1
Polymer preparation for salicylic acid

| Urea concentration | PAA | | | |
|---|---|---|---|---|
| for modification of | Heating | Autoclaving | | Viscosity |
| PAA* (%) | Time | Time | pH | (cP) |
| 2.1 | (No heating) | 80 min | 9.04 | 38.3 |
| 2.2 | 1 hr 23 min | 80 min | 9.31 | 42 |
| 2.2 | 1 hr 23 min | 80 min | 9.26 | 17.5 |
| 2.2 | 1 hr 23 min | 80 min | 9.34 | 29.4 |
| 2.2 | 1 hr 23 min | 80 min | 9.08 | 42.3 |
| 2.2 | 1 hr 03 min | 80 min | 9.17 | 38.9 |
| 2.2 | 1 hr 23 min | 80 min | 9.22 | 52.8 |
| 2.2 | 1 hr 23 min | 80 min | 9.08 | 39.42 |
| 2.2 | 1 hr 23 min | 80 min | 9.16 | 40.8 |
| 2.2 | 1 hr 40 min | 80 min | 9.17 | 36.9 |
| 2.2 | 2 h | 80 min | 9.25 | 39.5 |
| 2.2 | 2 h | 80 min | 9.24 | 38.4 |
| 2.2 | 2 h | 80 min | 9.28 | 38.8 |
| 2.2 | 1 hr 20 min | 80 min | 9.08 | 36 |
| 2.2 | 1 hr 20 min | 80 min | 9.05 | 38.8 |
| 2.2 | (No heating) | 80 min | 9.16 | 37.8 |
| 3 | (No heating) | (No autoclaving) | 5.98 | 6.0 |
| 3 | 1 hr 20 min | 80 min | 9.24 | 32.1 |
| 3 | 1 hr 23 min | 80 min | 9.03 | 40.2 |
| 3 | 1 hr 23 min | 80 min | 9.07 | (Not done) |
| 3 | 1 hr 40 min | (No autoclaving) | 7.37 | 11.1 |
| 4 | 1 hr 20 min | 80 min | 9.23 | 29.4 |
| (none) | 1 hr 20 min | 80 min | 6.93 | 10.5 |
| (none) | (No heating) | (No autoclaving) | 6.06 | 26.6 |
| (none) | (No heating) | (No autoclaving) | 6.32 | 25.47 |
| (none) | (No heating) | (No autoclaving) | 6.09 | 26 |
| (none) | (No heating) | (No autoclaving) | 6.03 | 26 |
| (none) | (No heating) | (No autoclaving) | 6.04 | 29 |
| (none) | 1 hr 30 min | (No autoclaving) | 6.97 | 6.32 |
| (none) | 1 hr 40 min | (No autoclaving) | 6.94 | 6.8 |
| (none) | 1 hr 40 min | 80 min | 6.8 | 9.2 |
| 2.2* | 1 hr 23 min | 80 min | 9.37 | 16.7 |
| 3* | 1 hr 23 min | 80 min | 9.19 | 20.1 |

The PAA concentration was 0.33% except in the instances indicated by an asterisk, when the PAA concentration was 0.1%.

### Example 2

### Preparation of Dispersions Comprising Nanoparticles of Inclusion Complexes of Salicylic Acid Wrapped in Urea-Modified Polyacrylamide via the Organic Solvent Process

In this method, the urea modified-PAA polymer solution in water is prepared as described in Example 1 and salicylic acid dissolved in an organic solvent is added to the solution, followed by autoclaving.

The conditions used to prepare dispersions comprising nanoparticles of inclusion complexes of salicylic acid with urea-modified PAA polymers using the organic solvent process are presented in Table 2. In specific experiments, 150-250 ml of the solution prepared in Example 1 were transferred to a reaction flask having three openings, one attached to a condenser, a second for insertion of a homogenizer (for stirring), and a third for solvent addition. Salicylic acid (17.5 grams) was dissolved in approximately 200 ml methyl acetate (MA) and was transferred to an apparatus suitable for dropwise addition into the reaction flask. The reaction flask was heated to approximately 67° C. Then, the homogenizer was started (stirring rate of approximately 2000-10,000 Reynolds) and the salicylic acid solution was fed at a rate of 1-3 drops per second into the reaction flask. As the complex formed, the methyl acetate evaporated and was collected after it passed through the condenser. Subsequently, the dispersion underwent autoclave treatment. The impact of the conditions used in the solvent process on the resulting salicylic acid concentration, pH, viscosity, and appearance of the product, are presented in Table 2. HPLC analysis was done to determine the salicylic acid concentration in the dispersions. The measured salicylic acid concentration is also presented as a percent of the expected final concentration. Urea treatment of PAA was found to be necessary for preparing SA inclusion complexes with PAA. This was also true for inclusion complexes prepared with polyacrylic acid (0.5%) modified by urea (1%).

TABLE 2
Preparation of salicylic acid complexes (with solvent)

| Water Media | | | | Organic Media | | | | | |
|---|---|---|---|---|---|---|---|---|---|
| Characteristics | | | | Characteristics | | | | | |
| Polymer | | | | Composition | | | | | |
| and | | Y1 | | (g SA/ml | | Y2 | | | SA*** |
| treatment* | pH | (cP) | T ° C. | solution) | Time** | (cP) | pH | Appearance | (%) |
| 0.33% PAA, | 9.17 | 36.9 | 70 | 17.5 g/250 ml | 80 min | 42.2 | 4.55 | Transparent | 90.70 |
| 2.2% Urea; | | | | | | | | | |
| 80 min | | | | | | | | | |
| 0.33% PAA, | 9.37 | 51.6 | 70 | 17.5 g/250 ml | 180 min | 32.2 | 6 | Transparent | 93.80 |
| 2.2% Urea; | | | | | | | | | |
| 80 min | | | | | | | | | |
| 5% | | | | 17.5 g/250 ml | 110 min | 21 | 5.13 | Transparent | 90.80 |
| polyacrylic | | | | | | | | pale tea color | |
| acid | | | | | | | | | |
| 1.0% Urea; | | | | | | | | | |
| 80 min**** | | | | | | | | | |

*length of autoclave treatment for polymer modification (minutes)
**length of final autoclave treatment in minutes
***HPLC measurement of SA (% of the theoretical concentration)
****sample used for the dialysis study
Y1 Polymer viscosity;
Y2 Final viscosity

### Example 3

### Two-Step Organic Solvent-Free Process for Preparation of Dispersions Comprising Nanoparticles of Inclusion Complexes of Salicylic Acid Wrapped in Urea-Modified Polyacrylamide

In the two-step process, a solution of PAA and urea in water is prepared as described in Example 1 and autoclaved for about 80 min, and salicylic acid powder is added to the modified polymer solution and autoclaved for about 130-180 min.

The modified polyacrylamide polymer was obtained by reaction of 0.33% or 0.2% PAA with 3% or 2.2% urea. After autoclave, 7.0 grams salicylic acid powder were added for each 100 ml of polymer solution, and the mixture was autoclaved (113-115° C.; 1.50-1.65 atm) for about 130-180 min. The combination of heat and pressure was essential for the solvent-free process, since otherwise significant amounts of crystalline salicylic acid precipitate. Under these conditions, the use of PAA unmodified by urea and of certain polymers such as chitosan or polyvinyl alcohol (PVA) did not lead to the desired dispersions and resulted in the precipitation of salicylic acid

As shown in Table 3, the pH of the resulting dispersion containing the nanoparticles of salicylic acid-polymer complexes ranged 4.46-7.94. Dispersions of salicylic acid with such pHs are suitable for formulations applicable to a variety of routes of administration, including oral, topical, and ocular routes. While the pH of formulations for oral administration is not limited, preparations with a neutral pH are preferred for ocular application and the more acidic preparations are preferred for topical application for skin treatment. The urea concentration was decreased to 2% and the PAA concentration was decreased to 0.18% and the resulting solutions of salicylic acid-polymer complexes had pH values of 4.73 and 4.8 (Table 3, last two rows). Precipitation was found to occur in dispersions having a pH below 4.5. Thus, the combination 0.18-0.2% PAA and 2% urea for the preparation of the modified polymer was found to be more suitable for the preparation of the salicylic acid inclusion complexes for topical use. The final salicylic acid concentration in the range of 58.52-70.56 mg/ml Table 3) was close to the theoretical (original) concentration value (70 mg/ml).

TABLE 3
Two-step solvent-free preparation of salicylic acid complexes

| | | | pH of the | |
|---|---|---|---|---|
| | | pH of Modified | SA-polymer | SA Conc. |
| % PAA | % Urea* | Polymer | complex | (mg/ml)** |
| 0.33 | 3 | 9.01 | 7.72 | 70.56 |
| 0.33 | 3 | 9.16 | 7.94 | 62.26 |
| 0.2*** | 2.2 | 9.19 | 4.92 | 66.6 |
| 0.2 | 2.2 | 9.18 | 6.46 | 66.82 |
| 0.33 | 2.2 | 8.97 | 6.56 | 69.02 |
| 0.33 | 2.2 | 9.1 | 6.39 | 66.99 |
| 0.2 | 3 | 9.17 | 7.94 | 68.35 |
| 0.2 | 2.2 | 9.19 | 4.92 | 66.6 |
| 0.2 | 2.2 | 9.18 | 6.46 | 66.82 |
| 0.2 | 2.2 | 9.24 | 4.87 | 61.73 |
| 0.2 | 2.2 | 9.24 | 5.16 | 67.01 |
| 0.2 | 2.2 | 9.26 | 4.46 | 58.52 |
| 0.2 | 2.2 | 9.26 | 7.11 | 68.35 |
| 0.18 | 2 | 9.05 | 4.73 | 68.28 |
| 0.18 | 2 | 9.05 | 4.8 | 62.27 |

*concentration of urea (%) for treating PAA;
**HPLC measurement of SA in the produced inclusion complexes (mg/ml);
***sample was examined by cryo-TEM

### Example 4

### One-Step Organic Solvent-Free Process for Preparation of Dispersions Comprising Nanoparticles of Inclusion Complexes of Salicylic Acid Wrapped in Urea-Modified Polyacrylamide

In the one-step process, a solution of PAA and urea in water is prepared as described in Example 1, salicylic acid powder is added to the modified polymer solution and autoclaved for about 130-180 min.

The amounts of the reagents and the reaction conditions are similar to the final conditions of Example 3 above. Thus, 0.2 grams polyacrylamide and 2.0 or 2.1 grams of urea were added per 100 ml water, and the mixture was heated to 95° C. while stirring, to form a solution having a pH ranging between 7.42-7.6. Then, approximately 7 grams of salicylic acid powder were added per 100 ml and the mixture was autoclaved (113-115° C.; 1.50-1.65 atm) for about 130-180 min. The complexes were formed during autoclave treatment of the mixture.

The conditions and results are shown in Table 4. No salicylic acid precipitated in these dispersions, as reflected by the low solution turbidity (Y1) and the concentration of salicylic acid in solution within the range 58.94-69.03 mg/ml (Y2). Furthermore, the pH of the final complexes within the range 4.38-5.89 (Y3) was suitable for topical application (about 4.7). The turbidity of the solutions was measured with a SMART2 colorimeter (LaMotte Company, Chestertown, Mass., USA) within a scale of 0-400 FTU (formazin turbidity unit). The results for turbidity shown in Table 4 (Y1: from 0 to 4) are for very clear solutions.

Attempts to shorten the autoclave treatment step by 10 minutes or more resulted in subsequent salicylic acid precipitation.

### Example 5

### Physical Analyses of Dispersions Comprising Nanoparticles of Inclusion Complexes of Salicylic Acid

(i) Particle Size Analyses

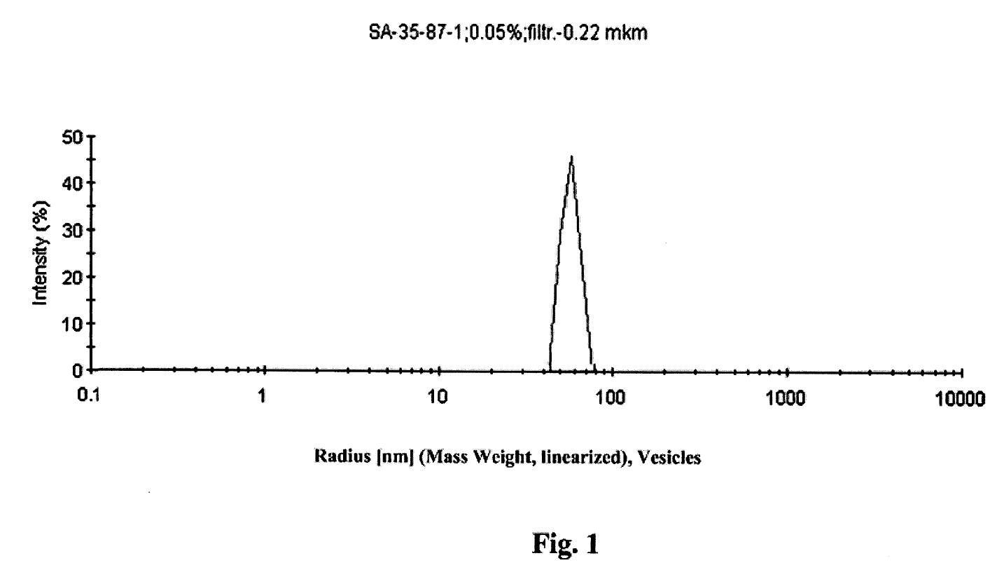

The size of nanoparticles of inclusion complexes of salicylic acid was analyzed using two methods, light scattering and cryo-transmission electron microscopy (TEM). Light scattering measurements of the nanoparticles size were performed using a Zetasizer Nano (Malvern Instruments, Ltd, Worcestershire, United Kingdom), which has a resolution of 0.6-6000 nm. Zetasizer Nano is a dynamic light scattering technique used to estimate the mean particle size. Dispersions comprising nanoparticle prepared as described in Examples 2 and 4 were measured using this method. A 1:10 dilution of the samples was found necessary for sample analysis. A typical graph of the particle size distribution, depicted in FIG. 1 (sample SA-35-87-1, of Table 4, Trial 4), shows that the diameters of the particles in the dispersions are typically about 50 nm. The narrow peaks obtained by these measurements indicate the high uniformity of the nanoparticle sizes in the dispersions.

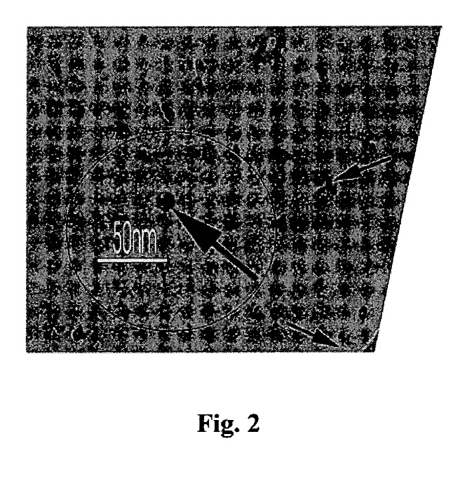

Cryo-TEM was also used to measure the size of the nanoparticles of inclusion complexes of salicylic acid. A sample prepared in Example 3 (as identified in Table 3) was examined by this method and the result shown in FIG. 2 demonstrates that the diameter of the salicylic acid nanoparticles is typically smaller than 50 nm. Therefore, both the one-step and two-step solvent-free methods yield dispersions having nanoparticles with similar sizes.

(ii) FTIR Analyses

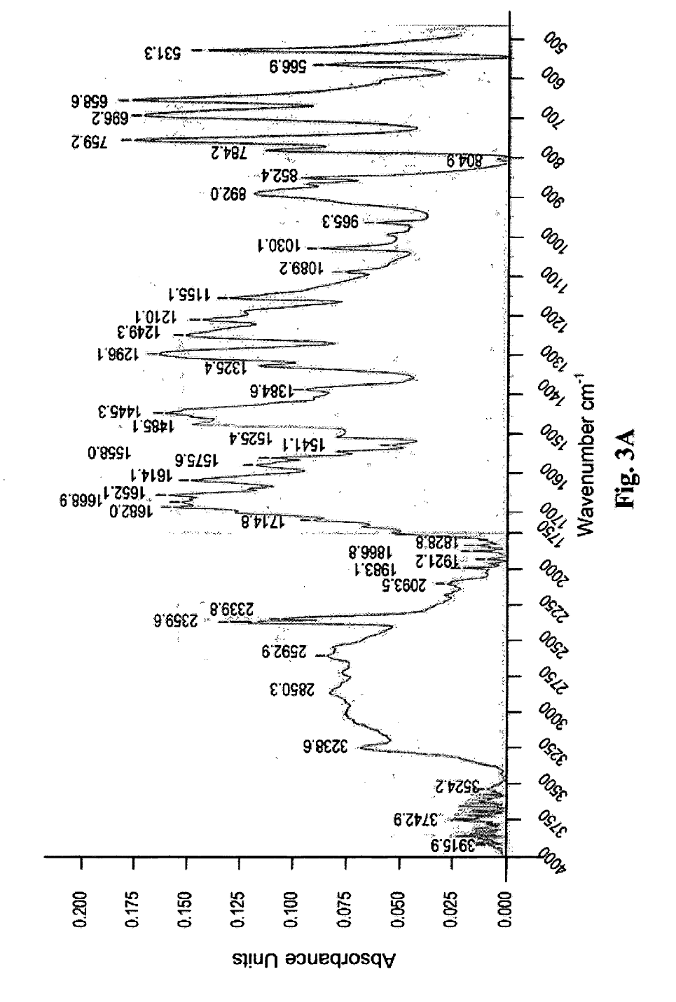

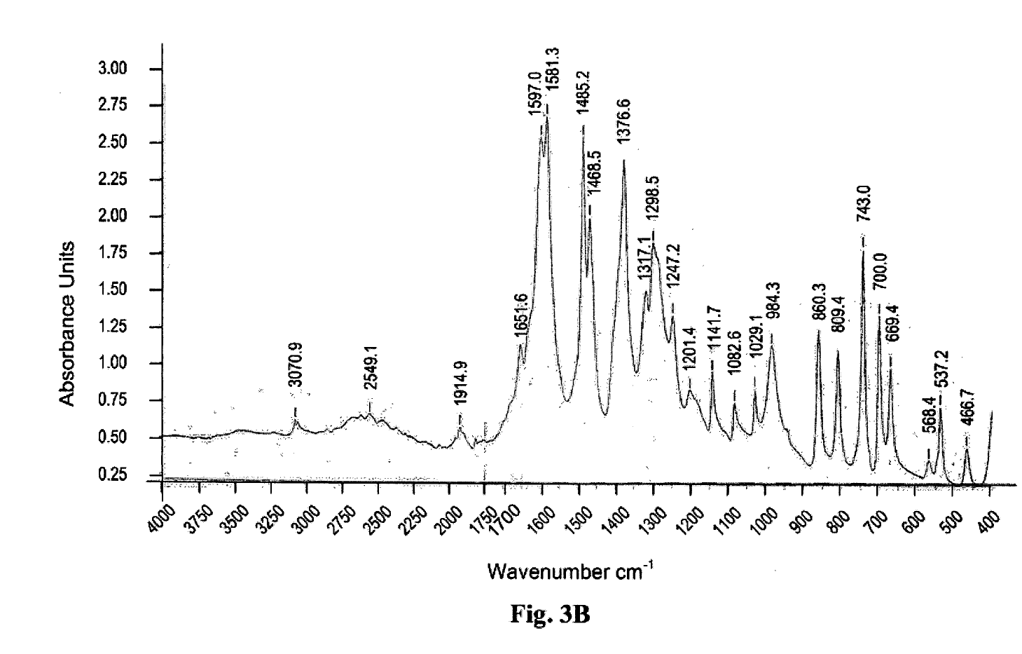

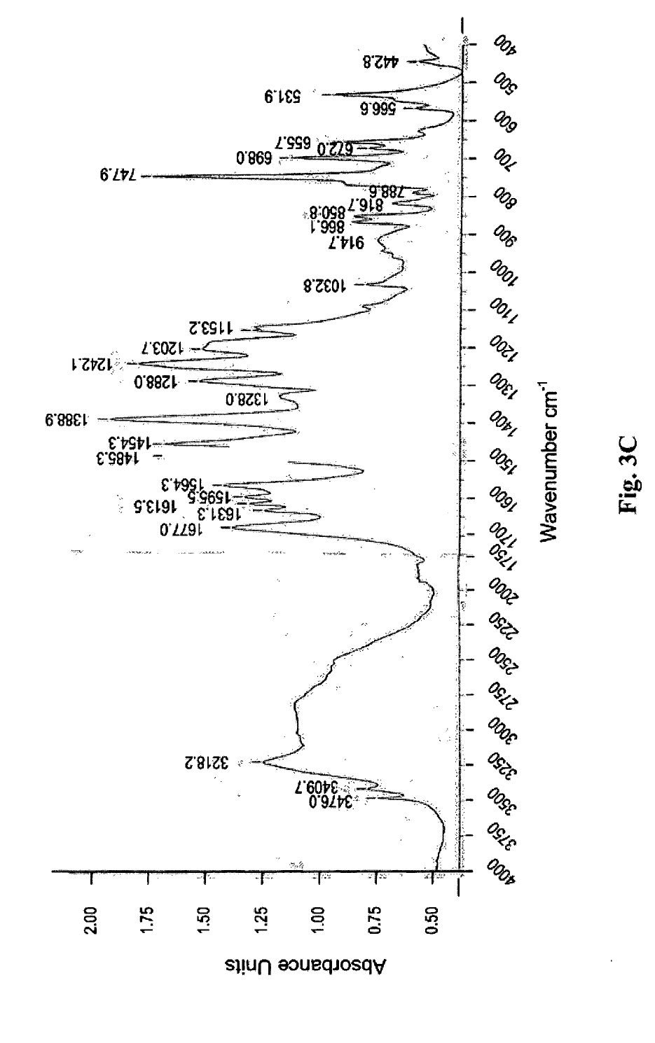

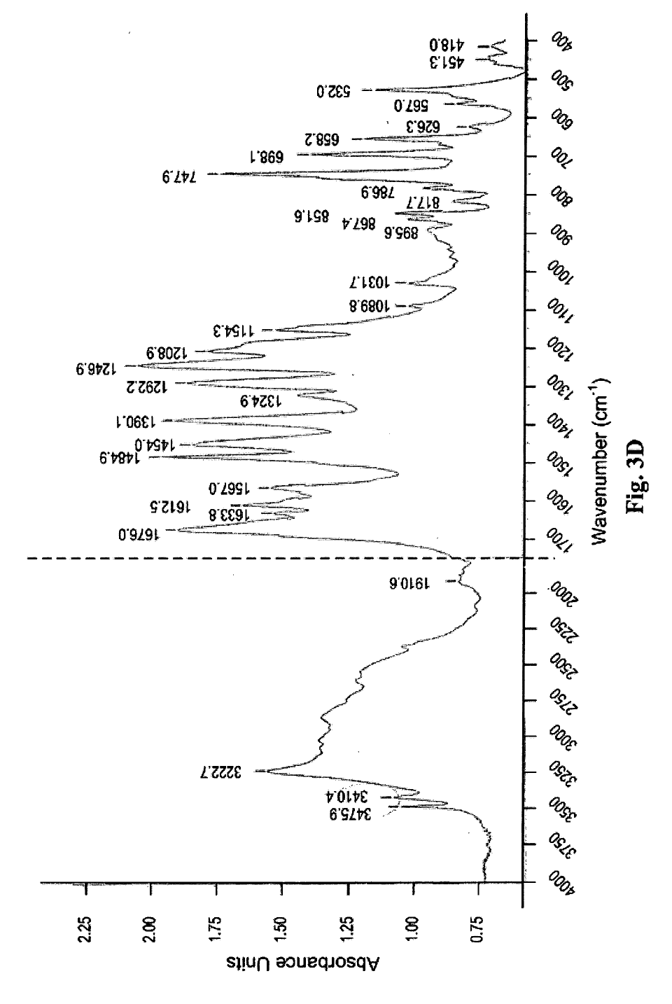

Fourier transform infrared (FTIR) spectroscopy analysis was performed for inclusion complexes of salicylic acid (7%) which were prepared by the two-step (35-57-2, prepared with 0.18% polyacrylamide and 2% urea) and one-step (35-87-2, prepared with 0.2% polyacrylamide and 2.1% urea) organic solvent-free processes. nThe results are shown in FIGS. 3A-3D, in which FIG. 3A depicts the infrared analysis of pure salicylic acid, FIG. 3B depicts the infrared analysis of the sodium salicylate salt (prepared by mixing salicylic acid with an approximately equimolar amount of NaOH, final pH 10), and FIGS. 3C-3D depict the absorbance profiles of salicylic acid nanoparticles prepared by the two-step process (sample 35-57-2) and the one-step process, respectively, It is to be noted that FIG. 3C and FIG. 3D are essentially identical. A summary comparison of the outstanding points of these absorbance profiles is presented in Table 5. The peak at 1581.3, that is unique for the salt, can be attributed to carboxylate anion stretching. The observation that this peak is not found in the spectra of the nanoparticles indicates that these inclusion complexes are not salicylic acid salts. Additionally, there is a focused peak at 1676.6, that is associated with the inclusion complexes and is more diffuse for pure salicylic acid. This may be the result of directed carbonyl stretching in the complexes.

### Example 6

### Release of Salicylic Acid from Nanoparticles Comprising Inclusion Complexes of Salicylic Acid

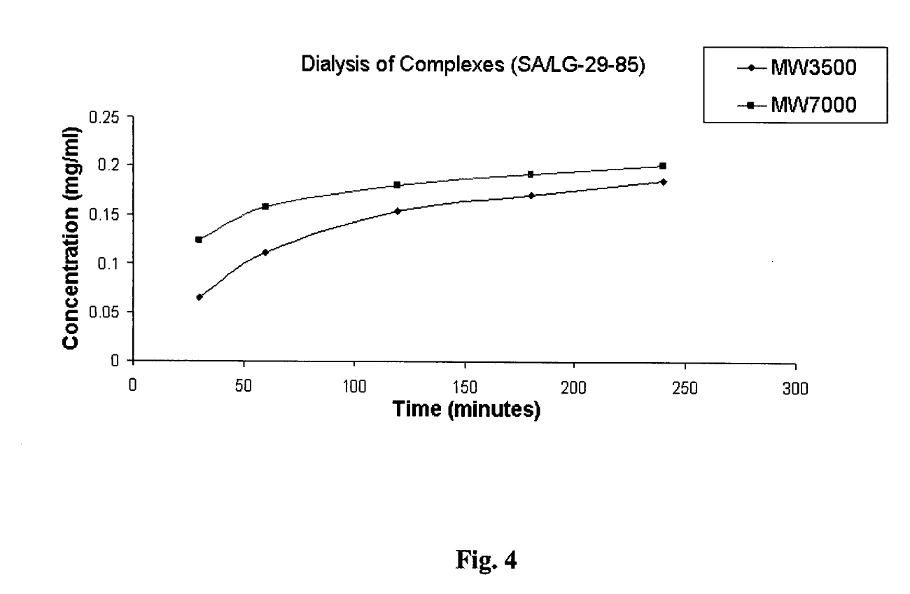

Release of salicylic acid from the inclusion complexes was assessed in vitro by monitoring changes in the salicylic acid concentration following salicylic acid passage through dialysis tubing. Dialysis tubing (Spectra/Por) having a pore size of either 3500 Daltons or 7000 Daltons was used, since the polymer is significantly greater than 7000 Daltons. The tubing was filled with 2 ml of a dispersion of nanoparticles (sample SA/LG-29-85, prepared using the two-step method with 1% or 2% urea and 0.2% PAA concentration, as described in Example 3) having a final salicylic acid concentration of 7%. The filled tubing was suspended in a beaker that contained 100 ml of alcohol (external solution). The external solution was continuously stirred in order to maintain a homogeneous salicylic acid concentration. At the times indicated in FIG. 4, 1 ml aliquots were removed from the external solution for analysis of the salicylic acid content by reverse-phase high pressure liquid chromatography (RP-HPLC). This analysis entailed preparation of a standard salicylic acid curve in which there was a linear relation between the salicylic acid concentration and the area of the measured salicylic acid samples of the curve. Measurement of the salicylic acid in each experimental sample was followed by calculation of the salicylic acid concentration according to the area of salicylic acid peak in that sample.

The results in FIG. 4 demonstrate that different rates of release were obtained when dialysis tubing with different pore sizes was used. The initial dialysis rate was faster when the pore size was larger. However, by four hours, the salicylic acid concentration in the external solution was similar in both experiments such that approximately 14% of the salicylic acid had migrated through the membrane. At this time point, in both cases, the salicylic acid concentration is still one tenth of its normal maximal solubility. Thus, the inclusion complexes provide a system that modifies salicylic acid release.

TABLE 4
Conditions for preparation of salicylic acid (SA)
nanoparticles by the one-step solvent-free method

| Trial | X1 | X2 | pH PAA-U | X3 | Y1 | Y2 | Y3 |
|---|---|---|---|---|---|---|---|
| 1 | 0.2 | 2.1 | 7.58 | 1 hr 50 min | 4 | 61.88 | 4.45 |
| 2 | 0.2 | 2.1 | 7.58 | 2 hr | 1 | 69.03 | 4.38 |
| 3 | 0.2 | 2.1 | 7.58 | 2 hr 10 min | 0 | 61.27 | 4.68 |
| 4* | 0.2 | 2.1 | 7.6 | 2 hr 10 min | 0 | 73.24 | 4.82 |
| 5 | 0.2 | 2.0 | 7.6 | 3 hr | 0 | 58.94 | 5.89 |
| 6 | 0.2 | 2.1 | 7.6 | 2 hr 20 min | 0 | 63.71 | 5.11 |

X1 - Concentration polyacrylamide (PAA)
X2 - Concentration urea (U)
X3 - Time of autoclaving complex
Y1 - Turbidity (FTU, scale 0-400)
Y2 - HPLC analysis (SA mg/ml)
Y3 - pH Complex SA/PAA-U
*Sample Sa-35-87-1

TABLE 5
FTIR analysis of salicylic acid (SA), salicylate salt
and SA nano-particles

| | Wavelength (cm⁻¹) | | | |
|---|---|---|---|---|
| Sample | 2500-3500 | 1676.6 | 1581.3 | 1454 |
| SA | + | + | − | + |
| | | (1682-1652) | | (1485) |
| Sodium salicylate salt | − | − | + | − |
| SA nano-particles | + | + | − | + |
| (2-step process*) | | | | |
| SA nano-particles | + | + | − | + |
| (1-step process*) | | | | |

*7% SA dispersions prepared with 0.33% polyacrylamide modified with 2% urea

1. A hydrophilic inclusion complex consisting essentially of nanosized particles of salicylic acid wrapped in an amphiphilic polymer such that non-valent bonds are formed between the salicylic acid and the amphiphilic polymer, wherein said amphiphilic polymer is selected from the group consisting of polyacrylic acid, polyacrylamide and copolymers thereof, polymethacrylamide and copolymers thereof, and polylysine, and said amphiphilic polymer is modified by reaction with urea or a derivative thereof, nicotinamide or guanidine.

2. The hydrophilic inclusion complex according to claim 1, wherein said amphiphilic polymer is a copolymer of acrylamide or methacrylamide with one or two monomers selected from the group consisting of acrylic acid, methacrylic acid, an alkyl acrylate, an alkyl methacrylate, acrylonitrile, ethyleneimine, vinyl acetate, styrene, maleic anhydride and vinyl pyrrolidone, and said amphiphilic polymer is modified by reaction with urea or a derivative thereof, nicotinamide or guanidine.

3. The hydrophilic inclusion complex according to claim 1, wherein said amphiphilic polymer is modified by reaction with urea or a urea derivative selected from the group consisting of methylol urea, acetyl urea, semicarbazide and thiosemicarbazide.

4. The hydrophilic inclusion complex according to claim 3, wherein said amphiphilic polymer is polyacrylamide modified by reaction with urea.

5. The hydrophilic inclusion complex according to claim 3, wherein said amphiphilic polymer is polymethacrylamide modified by reaction with urea.

6. A hydrophilic dispersion comprising nanoparticles of inclusion complexes of salicylic acid wrapped in an amphiphilic polymer such that non-valent bonds are formed between the salicylic acid and the amphiphilic polymer, wherein said amphiphilic polymer is selected from the group consisting of polyacrylic acid, polyacrylamide and copolymers thereof, polymethacrylamide and copolymers thereof, and polylysine, and said amphiphilic polymer is modified by reaction with urea or a derivative thereof, nicotinamide or guanidine.

7. The hydrophilic dispersion according to claim 6, wherein said amphiphilic polymer is a copolymer of acrylamide or methacrylamide with one or two monomers selected from the group consisting of acrylic acid, methacrylic acid, an alkyl acrylate, an alkyl methacrylate, acrylonitrile, ethyleneimine, vinyl acetate, styrene, maleic anhydride and vinyl pyrrolidone, and said amphiphilic polymer is modified by reaction with urea or a derivative thereof, nicotinamide or guanidine.

8. The hydrophilic dispersion according to claim 7, wherein said amphiphilic polymer is modified by reaction with urea or a urea derivative selected from the group consisting of methylol urea, acetyl urea, semicarbazide and thiosemicarbazide.

9. The hydrophilic dispersion according to claim 8, wherein said amphiphilic polymer is polyacrylamide modified by reaction with urea.

10. The hydrophilic dispersion according to claim 8, wherein said amphiphilic polymer is polymethacrylamide modified by reaction with urea.

11. The hydrophilic dispersion according to claim 6, wherein said nanoparticles are in the range of from approximately 10 to approximately 100 nanometers in size.

12. A process for the preparation of a dispersion of salicylic acid nanoparticles according to claim 6, which comprises:
(i) preparation of a solution of the amphiphilic polymer in water;
(ii) modification of the amphiphilic polymer by reaction with urea or a derivative thereof, nicotinamide or guanidine, under heat and pressure;
(iii) preparation of a molecular solution of salicylic acid in an organic solvent;
(iv) dripping the cold organic solution (iii) of the salicylic acid into the modified polymer water solution (ii), while heating at a temperature 5-10° C. above the boiling point of the organic solvent of (iii), under constant mixing, thus causing evaporation of the organic solvent; and
(v) subjecting the dispersion obtained in (iv) to autoclave treatment, thus obtaining the desired dispersion comprising nanoparticles of inclusion complexes of salicylic acid entrapped within said amphiphilic polymer.

13. The process according to claim 12, wherein the amphiphilic polymer is polyacrylamide modified by reaction with urea.

14. The process according to claim 12, wherein said solvent is methyl acetate or dichloromethane.

15. A two-step solvent-free process for the preparation of a dispersion comprising salicylic acid nanoparticles according to claim 6, comprising:
(i) preparation of a solution of the amphiphilic polymer in water;
(ii) modification of the amphiphilic polymer by reaction with urea or a derivative thereof, nicotinamide or guanidine, under heat and pressure, in an autoclave;
(iii) addition of salicylic acid powder to the modified polymer water solution; and
(iv) subjecting the dispersion obtained in (iii) to autoclave treatment, thus obtaining the desired dispersion comprising nanoparticles of inclusion complexes of salicylic acid entrapped within said modified amphiphilic polymer.

16. The process according to claim 15, wherein the amphiphilic polymer is polyacrylamide modified by reaction with urea.

17. A one-step solvent-free process for the preparation of a dispersion comprising salicylic acid nanoparticles according to claim 6, comprising:
(i) preparation of a solution of the amphiphilic polymer in water;
(ii) modification of the amphiphilic polymer by reaction with urea or a derivative thereof, nicotinamide or guanidine;
(iii) addition of salicylic acid powder to the modified polymer water solution; and
(iv) subjecting the dispersion obtained in (iii) to autoclave treatment, thus obtaining the desired hydrophilic dispersion comprising nanoparticles of inclusion complexes of salicylic acid entrapped within said amphiphilic polymer.

18. The process according to claim 17, wherein the amphiphilic polymer is polyacrylamide modified by reaction with urea.

19. A stable pharmaceutical composition comprising a pharmaceutically acceptable carrier and a hydrophilic dispersion of salicylic acid nanoparticles according to claim 6.

20. A stable cosmetic composition comprising a hydrophilic dispersion of salicylic acid nanoparticles according to claim 6.
